(19) United States
(12) Patent Application Publication
(10) Pub. No.: US 2010/0240621 A1
(43) Pub. Date: Sep. 23, 2010

(54) TOPICAL PHARMACEUTICAL COMPOSITION FOR THE COMBINATION OF FUSIDIC ACID AND A CORTICOSTEROID

(75) Inventors: Nilendu Sen, Mumbai (IN); Kusum Dinkar Gole, Mumbai (IN); Akhilesh Dayanand Sharma, Mumbai (IN)

Correspondence Address: GLENMARK GENERICS INC., 750 CORPORATE DRIVE, MAHWAH, NJ, 07430, US

(73) Assignee: Glenmark Pharmaceuticals Ltd., Mumbai (IN)

(21) Appl. No.: 12/677,298

(22) PCT Filed: Sep. 8, 2008
(86) PCT No.: PCT/IN08/00577

§ 371 Date: Mar. 9, 2010

(30) Foreign Application Priority Data
Sep. 10, 2007 (IN) 1725/MUM/2007

(51) Int. Cl. A61K 31/58; A61P 17/00; A61P 31/04

(52) U.S. Cl. 514/170

(57) ABSTRACT

The present invention relates to topical pharmaceutical compositions comprising fusidic acid and a corticosteroid. More particularly, the present invention relates to topical pharmaceutical compositions comprising fusidic acid and mometasone or halobetasol or their esters, processes for preparing the same, and the use of such compositions for prevention and treatment of dermal infections.

### PRIORITY DETAILS

This patent application claims priority to Indian Patent Application No. 1725/MUM/2007, filed on Sep. 10, 2007, the contents of which are hereby incorporated as reference.

### TECHNICAL FIELD OF THE INVENTION

The present invention relates to a combination therapy of a topical antibiotic and a topical steroid for the treatment of inflammatory dermatoses associated with secondary bacterial infections. In particular the present invention relates to topical pharmaceutical compositions comprising a combination of fusidic acid and corticosteroid such as Mometasone furoate useful in treatment of infected eczema's such as secondarily infected dermatitis, including secondarily infected contact dermatitis, psoriasis, allergic contact dermatitis and atopic dermatitis with secondary bacterial infections of skin. In particular the present invention also relates to topical pharmaceutical compositions comprising a combination of fusidic acid and corticosteroid such as Mometasone furoate useful in prevention of infection in cases of dermatitis, especially atopic dermatitis who are at risk of getting secondary bacterial infection

In particular the present invention also relates to topical pharmaceutical compositions comprising a combination of fusidic acid and corticosteroid such as Halobetasol propionate useful in treatment of infected steroid responsive dermatoses such as secondarily infected dermatoses including secondarily infected contact dermatitis, allergic contact dermatitis, atopic dermatitis, psoriasis and other corticosteroid responsive dermatoses (CRD) with secondary bacterial infections of skin. In particular the present invention also relates to topical pharmaceutical compositions comprising a combination of fusidic acid and corticosteroid such as Halobetasol propionate useful in prevention of secondary bacterial infections in patients with non-infected dermatoses

### BACKGROUND OF THE INVENTION

Bacteria such as Staphylococcus can live harmlessly on many skin surfaces, but when the skin is punctured or broken for any reason, staphylococcus bacteria can enter the wound and cause the infections. There are more than 30 species in the staphylococcus family of bacteria, and they can cause different kinds of illnesses. But most staphylococcus infections are caused by the species Staphylococcus aureus (S. aureus) and Streptococcus pyogenes. Skin and soft-tissue infections are among the most common infections, and may lead to serious local and systemic complications. Bacterial infections occur frequently in lesions of eczema and atopic dermatitis. One of the common causes of skin and soft-tissue infections is the occurrence of secondary bacterial infection that complicates skin lesions. Secondary bacterial skin infections are common complications of primary dermatoses, primary nonbacterial skin infections, traumatic lesions, ulcers, cutaneous infestations, and other miscellaneous skin diseases. Despite the differences in the underlying cause and clinical presentation, overt secondary bacterial infection with Staphylococcus aureus and Streptococcus pyogenes, or both is a common problem in patients with inflammatory skin diseases such as allergic contact dermatitis, atopic dermatitis, or psoriasis. Various host factors have been suggested to be the cause of secondarily infected dermatitis (SID), including the lack of expression of antimicrobial peptides on the skin, and the demonstration of increased adherence of Staphylococcus aureus to the skin of patients with atopic dermatitis, A serious consequence of atopic dermatitis, psoriasis, and allergic contact dermatitis is that the integrity of the skin barrier is compromised. Recent studies of patients with atopic dermatitis have shown that levels of both epidermal hydration and skin surface lipids are reduced, making the skin more susceptible to colonization with pathogens.

A correlation between the severity of the eczema and colonization with S. aureus has been demonstrated, and it has been determined that bacterial colonization is an important factor aggravating skin lesions. Staphylococcus aureus has also been found on the skin of more than half of the patients with chronic plaque psoriasis, and colonization with staphylococci and streptococci has been reported to exacerbate psoriasis. Staphylococcal superantigens contribute to the pathogenesis of cutaneous inflammation in atopic dermatitis through various potential mechanisms, viz. direct stimulation of antigen presenting cells and keratinocytes, stimulation of T cell proliferation (by binding to T cell receptors), expansion of skin-homing cutaneous lymphocyte antigen positive T cells. Topical corticosteroid is commonly used in the treatment of eczema and atopic dermatitis and several studies have shown the impact of such treatment on bacterial skin flora.

Antibacterials, whether topical or systemic, in combination with topical corticosteroids, may produce a more rapid decrease in S. aureus colonization than topical corticosteroids monotherapy. Corticosteroids are commonly used in treatment of eczema and Atopic dermatitis. However, when steroid application or administration is stopped, the diseases easily relapse, and when it is used for a long time it may lead to a number of adverse effects such as skin atrophy and secondary infection. Bacterial superantigens were recently shown to induce corticosteroid insensitivity. Hence the eradication of Staphylococcus aureus may lead to a steroid-saving effect.

Leyden et al. (Br J Dermatol 1974; 90:525-30.) first showed that the therapeutic effect of antibiotics combined with steroid was better than that of steroid monotherapy. In several open or double-blind placebo-controlled trials, topical and systemic antimicrobials were able to reduce the colonization density and led to a partial improvement of skin lesions.

Larsen FS et al (Acta Derm Venereol. 2007; 87 (1):62-8.) provides a formulation of fusidic acid and betamethasone 17-valerate as an alternative for the short-term treatment of clinically infected atopic dermatitis.

M. A. Cobb (The Veterinary Journal Volume 169, Issue 2, March 2005, Pages 276-280) et al provides a comparative study of topical preparation containing 0.5% fusidic acid and 0.1% betamethasone-17-valerate with the systemic therapy. The study demonstrates no difference and that both treatment regimes represent effective treatment options for the condition such as canine acute moist dermatitis.

Strategos J. (Pharmatherapeutica. 1986; 4(9):601-6.) provides a comparison study of Fusidic acid-betamethasone combination and gentamicin-betamethasone combination.

U.S. Pat. No. 6,673,783 assigned to Leo Pharmaceutical Products Ltd, provides compounds of Fusidic acid derivatives. The invention also provides combination of Fusidic acid derivatives with other therapeutically active components such as penicillins, cephalosporins, tetracyclines, rifamycins, erythromycins, linocomycin, clindamycin, fluoroquinolones and corticosteroids.

WO2007051468 assigned to Leo Pharma, provides the pharmaceutical composition comprising crystalline fusidic acid. The patent application also provides compositions further comprising another therapeutically active compound selected from the group consisting of antibiotics and corticosteroids.

U.S. Pat. No. 6,127,353 assigned to Schering Corporation provides mometasone furoate monohydrate and its pharmaceutical compositions.

Combination therapy of a potent topical steroid and a topical antibiotic has been proved to be effective in a number of clinical studies. The secondary bacterial infections can occur as a side effect during long term therapy with potent steroids. These secondary bacterial infections also exacerbate the underlying inflammatory dermatoses, further increasing the amount of steroid required to control it. Advantages of the topical route for antibacterial therapy include the ability to deliver a higher concentration of antibacterial to the skin than would be possible with systemic therapy. Its use also mostly avoids severe systemic reactions.

However, the above mentioned prior arts fail to disclose the composition comprising a combination of fusidic acid with corticosteroids especially Mometasone or Halobetasol. Therefore a need remains to develop the topical preparations comprising the combination of fusidic acid with corticosteroids especially Mometasone or Halobetasol. The present invention provides pharmaceutical compositions comprising a combination of fusidic acid and mometasone furoate, which seems to be novel and a good rationale for the treatment of inflammatory dermatoses associated with secondary bacterial infections. The inventors of the present invention surprisingly found that antibiotic action of fusidic acid and the anti-inflammatory effect of corticosteroid, such as Mometasone both play important roles in reducing S. aureus and improving patient's symptoms and signs of skin inflammatory infections. Preferably, a combination of fusidic acid and mometasone furoate is useful for the treatment of inflammatory dermatoses associated with secondary bacterial infections, where the dermatoses is taken care of by mometasone and the bacterial infection is treated with fusidic acid.

The present invention provides pharmaceutical compositions comprising a combination of fusidic acid and Halobetasol propionate. Combination therapy of a potent topical steroid and a topical antibiotic has been proved to be effective in a number of clinical studies. The secondary bacterial infections can occur as a side effect during long term therapy with potent steroids. These secondary bacterial infections also exacerbate the underlying inflammatory dermatoses, further increasing the amount of steroid required to control it. Staphylococcus aureus and streptococcus pyogenes are the most common organisms infecting the inflammatory dermatoses, hence a combination of Halobetasol propionate and Fusidic acid seems to be a good rationale for the treatment of inflammatory dermatoses associated with secondary bacterial infections, where the dermatoses is taken care of by Halobetasol propionate and the bacterial infection is treated with fusidic acid. The inventors of the present invention surprisingly found that antibiotic action of fusidic acid and the anti-inflammatory effect of a corticosteroid such as Halobetasol, both play important roles in the prevention of secondary bacterial infections in patients with non-infected dermatoses and in treatment of infected steroid responsive dermatoses such as secondarily infected dermatoses including secondarily infected contact dermatitis, allergic contact dermatitis, atopic dermatitis, psoriasis and other corticosteroid responsive dermatoses (CRD) with secondary bacterial infections of skin.

### OBJECT OF THE INVENTION

An object of the present invention is to provide a topical pharmaceutical compositions comprising combination of a topical antibiotic and a topical steroid for the treatment of inflammatory dermatoses associated with secondary bacterial infections. In particular the present invention relates to topical pharmaceutical compositions comprising a combination of fusidic acid and corticosteroid such as Mometasone furoate useful in treatment of infected eczema's such as secondarily infected dermatitis, including secondarily infected contact dermatitis, psoriasis, allergic contact dermatitis and atopic dermatitis with secondary bacterial infections of skin. In particular the present invention also relates to topical pharmaceutical compositions comprising a combination of fusidic acid and corticosteroid such as Mometasone furoate useful in prevention of infection in cases of dermatitis, especially atopic dermatitis who are at risk of getting secondary bacterial infection

In particular the present invention also relates to topical pharmaceutical compositions comprising a combination of fusidic acid and corticosteroid such as Halobetasol propionate useful in treatment of infected steroid responsive dermatoses such as secondarily infected dermatoses including secondarily infected contact dermatitis, allergic contact dermatitis, atopic dermatitis, psoriasis and other corticosteroid responsive dermatoses (CRD) with secondary bacterial infections of skin. In particular the present invention also relates to topical pharmaceutical compositions comprising a combination of fusidic acid and corticosteroid such as Halobetasol propionate useful in prevention of secondary bacterial infections in patients with non-infected dermatoses

### SUMMARY OF THE INVENTION

One embodiment of the present invention provides a topical pharmaceutical composition in a suitable dosage form comprising the combination of therapeutically effective amount of an antibiotic, therapeutically effective amount of corticosteroid and a pharmaceutically acceptable carrier. Suitable dosage forms include hydrous or anhydrous semisolids such as creams, gels, ointments, lotions and the like.

Another embodiment of the present invention provides the topical pharmaceutical compositions comprising the combination of a therapeutically effective amount of antibiotic agent such as fusidic acid and therapeutically effective amount of corticosteroid such as Mometasone furoate or any other pharmaceutically acceptable salts/solvates of Mometasone and a pharmaceutically acceptable carrier thereof.

Another embodiment of the present invention provides the use of topical pharmaceutical dosage forms comprising the combination of therapeutically effective amount of antibiotic agent such as fusidic acid and therapeutically effective amount of corticosteroid such as Mometasone furoate or any other pharmaceutically acceptable salts/solvates of Mometasone for the treatment of infected eczema's such as secondarily infected dermatitis, including secondarily infected contact dermatitis, allergic contact dermatitis, psoriasis and atopic dermatitis with secondary bacterial infections of skin.

Another embodiment of the present invention provides the use of topical pharmaceutical dosage forms comprising the combination of therapeutically effective amount of antibiotic agent such as fusidic acid and therapeutically effective amount of corticosteroid such as Mometasone furoate or any other pharmaceutically acceptable salts/solvates of Mometasone useful in the prevention of infection in cases of dermatitis, specially atopic dermatitis who are at risk of getting secondary bacterial infection

Another embodiment of the present invention provides the topical pharmaceutical compositions comprising the combination of therapeutically effective amount of combination of a) 1% w/w to 5% w/w of fusidic acid; b) 0.05% w/w to 2% w/w of Mometasone furoate; and c) a pharmaceutically acceptable carrier.

Another embodiment of the present invention provides the use of topical pharmaceutical dosage forms comprising the combination of fusidic acid present in the concentration range of 1% w/w to 5% w/w and Mometasone furoate present in the concentration range of 0.05% w/w to 2% w/w for the treatment of infected eczema's such as secondarily infected dermatitis, including secondarily infected contact dermatitis, allergic contact dermatitis and atopic dermatitis with secondary bacterial infections of skin or in prevention of infection in cases of dermatitis, specially atopic dermatitis who are at risk of getting secondary bacterial infection

Another embodiment of the present invention provides the process for preparing topical pharmaceutical dosage forms comprising a combination of a) an effective amount of fusidic acid; and b) an effective amount of mometasone furoate; and c) a pharmaceutically acceptable carrier thereof. The topical compositions of the present invention having the weight ratio of Mometasone furoate and fusidic acid ranging from 1:2.5 to 1:20.

Suitable dosage forms include hydrous or anhydrous semisolids such as creams, gels, ointments, creams, lotions or any other dosage form suitable for topical application and the like.

Another embodiment of the present invention provides the topical pharmaceutical compositions comprising the combination of a) a therapeutically effective amount of antibiotic agent such as fusidic acid; and b) a therapeutically effective amount of corticosteroid such as Halobetasol propionate or any other pharmaceutically acceptable salts/solvates of Halobetasol; and c) a pharmaceutically acceptable carrier thereof.

Another embodiment of the present invention provides the use of topical pharmaceutical dosage forms comprising the combination of therapeutically effective amount of antibiotic agent such as fusidic acid and therapeutically effective amount of corticosteroid such as Halobetasol propionate or any other pharmaceutically acceptable salts/solvates of Halobetasol in the treatment of infected steroid responsive dermatoses such as secondarily infected dermatoses including secondarily infected contact dermatitis, allergic contact dermatitis, atopic dermatitis, psoriasis and other corticosteroid responsive dermatoses (CRD) with secondary bacterial infections of skin. Another embodiment of the present invention provides the use of topical pharmaceutical dosage forms comprising the combination of therapeutically effective amount of antibiotic agent such as fusidic acid and therapeutically effective amount of corticosteroid such as Halobetasol propionate or any other pharmaceutically acceptable salts/solvates of Halobetasol useful in prevention of secondary bacterial infections in patients with non-infected dermatoses.

Another embodiment of the present invention provides the topical pharmaceutical compositions comprising the combination of therapeutically effective amount of a) 1% w/w to 5% w/w of fusidic acid; and b) 0.01% to 2% w/w of Halobetasol propionate; and c) a pharmaceutically acceptable carrier. The topical compositions of the present invention having the weight ratio of Halobetasol propionate and fusidic acid ranging from 1:2.5 to 1:100.

Another embodiment of the present invention provides the use of topical pharmaceutical dosage forms comprising the combination of fusidic acid present in the concentration range of 1% w/w to 5% w/w and Halobetasol propionate present in the concentration range of 0.01% to 2% for the treatment of infected steroid responsive dermatoses such as secondarily infected dermatoses including secondarily infected contact dermatitis, allergic contact dermatitis, atopic dermatitis, and other corticosteroid responsive dermatoses (CRD) with secondary bacterial infections of skin or in prevention of secondary bacterial infections in patients with non-infected dermatoses.

Another embodiment of the present invention provides the process for preparing topical pharmaceutical dosage forms comprising the combination of a) an effective amount of fusidic acid; b) an effective amount of Halobetasol propionate; and c) a pharmaceutically acceptable carrier thereof. Suitable dosage forms include hydrous or anhydrous semisolids such as creams, gels, ointments, creams, lotions or any other dosage form suitable for topical application and the like.

### DETAILED DESCRIPTION OF THE INVENTION

Before describing the present invention in detail, it is to be understood that this invention is not limited to specific pharmacologically active carriers, formulation types, treatments, and so forth, as such may vary. It is also to be understood that the terminology used herein is for the purpose of describing particular embodiments only, and is not intended to be limiting.

### DEFINITIONS

The term “therapeutically effective amount” or “effective amount” is used herein to denote any amount of a topical formulation which will cause a substantial improvement in a disease condition when applied to the affected area. A single application can be sufficient, or the formulation can be applied repeatedly over a period of time. The amount will vary with the condition being treated, the stage of advancement of the condition, and the type and concentration of formulation applied. In the present invention, antibiotic agent such as fusidic acid is present in the concentration range of 1% w/w to 5% w/w of the total composition and corticosteroid such as Mometasone furoate is present in the concentration range of 0.05% w/w to 2% w/w of the total composition. In the present invention, antibiotic agent such as fusidic acid is present in the concentration range of 1% w/w to 5% w/w of the total composition and corticosteroid such as Halobetasol propionate is present in the concentration range of 0.01% w/w to 2% w/w of the total composition.

The terms “drug” and “pharmaceutical” are also used interchangeably to refer to a pharmacologically active substance or composition.

The term “topical composition” or “topical formulation” means a composition in which the drug may be placed for direct application to a skin surface and from which an effective amount of the drug is released. Such formulations may include creams, ointments, gels, lotions, or any other dosage form suitable for topical application and the like. In some aspects, such formulations may be applied to the skin in an unoccluded form with/without additional backing, structures or devices.

The term “skin” or “skin surface” is meant to include the outer skin of a subject comprising one or more of epidermal layers to which a drug composition may be administered.

The term “treating” or “treatment” of a state, disorder or condition as used herein means: (1) preventing or delaying the appearance of clinical symptoms of the state, disorder or condition developing in a mammal.

The term “Eczema” also known as dermatitis is a non-infectious skin disorder characterized by itching and often accompanied by small blisters. It may be caused by a variety of internal (endogenous) & external (exogenous) factors and it may be acute or chronic. There are numerous types of eczema, it can be either Atopic dermatitis or Contact dermatitis.

Atopic dermatitis is a chronic skin disease, commonest form of eczema and is closely linked with asthma and hay fever and can affect both children & adults can also be inherited and usually appears in infancy or early childhood. This disease becomes worsen after eating certain foods or after being exposed to other allergens such as pollen or dust. Most common symptoms include itchiness (or pruritus), dryness of skin, redness & inflammation. Constant scratching can also cause skin to split, leaving it prone to infection and thus bacterial infections occur frequently in lesions of atopic dermatitis.

Contact dermatitis is a localized reaction that includes redness, itching, and burning. Contact dermatitis occurs when the skin has come into contact with allergens or irritants and produces either irritant contact dermatitis or allergic contact dermatitis. Irritant contact dermatitis is a direct irritation of skin caused due to direct chemical damage that releases mediators of inflammation predominately from epidermal cells. Allergic contact dermatitis is a red, itchy, weepy reaction when the skin has come into contact with a substance that the immune system recognizes as foreign, such as poison ivy, poison oak or poison sumac or certain preservatives present in creams and lotions. This type of reaction reflects a specific sensitivity or allergy to a specific substance. Because of damage to the skin barrier function they have a propensity of becoming secondarily infected with bacteria; infected eczema skin may crack & weep (‘wet’ eczema).

The term “pharmaceutically acceptable” such as in the recitation of a “pharmaceutically acceptable carrier” or a “pharmaceutically acceptable salt/solvate or derivative” is meant a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained.

The term “carriers” or “vehicles” as used herein refer to pharmaceutically acceptable carrier materials suitable for topical drug administration. Carriers and vehicles useful herein include any such materials known in the art that are non-toxic and do not interact with other components of the composition in a deleterious manner.

“Fusidic acid” is a steroidal antibiotic, chemically related to cephalosporin P. It interferes with amino acid transfer from amino aminoacyls RNA to protein on the ribosomes inhibiting bacterial protein synthesis. It may be bacteriostatic or bactericidal depending upon the drug concentration. Fusidic acid is active against gram-positive bacteria, particularly staphylococci, with almost no activity against gram-negative organisms. It is also active against Streptococci (including Pneumococci) and Corynebacteria. At least 99% of oxacillin-resistant staphylococcus aureus were inhibited at a concentration of 2 mg/L, 100% of oxacillin-sensitive staphylococcus aureus were inhibited at a concentration of 0.12 mg/L and 99% of staphylococcus epidermidis were inhibited at 4 mg/L. Other bacteria that demonstrated 90% of organisms inhibited at 2 mg/L or less included; Peptococcus species, Clostridium species Propionibacterium Danes, and most Bacteroides species. Fusidic acid 2% cream monotherapy or combination therapy (betamethasone, beclomethasone dipropionate, ketoconazole, and sodium fusidate) is effective in the treatment of secondary bacterial infections occurring in dermatitis, pyoderma, furuncle, eczema, burns, and psoriasis.

“Corticosteroids” are a class of steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior. Topical corticosteroids are used to help relieve redness, swelling, itching, and discomfort of many skin problems. These medicines are like cortisone. They belong to the general family of medicines called steroids. Steroidal anti-inflammatory agents, including but not limited to, corticosteroids such as mometasone, Fluticasone, Clobetasone, hydrocortisone, hydroxyltriamcinolone, alpha-methyl dexamethasone, dexamethasone-phosphate, beclomethasone dipropionate, alclometasone, clobetasol valerate, Desoximetasone, Diflorasone, Fluocinolone, Fluocinonide, Halobetasol, desonide, desoxycorticosterone acetate, dexamethasone, dichlorisone, deflorasonediacetate, diflucortolone valerate, fluadronolone, fluclarolone acetonide, fludrocortisone, flumethasone pivalate, fluosinolone acetonide, fluocionide, flucortine butylester, fluocortolone, flupredidene (flupredylidene) acetate, flurandronolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone and its esters, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone, triamcinolone and mixtures thereof may be used. Preferably, mometasone or halobetasol may be used.

“Mometasone” a halogenated monoester, is a synthetic corticosteroid which is highly effective but may have a lower incidence of adverse effects than other corticosteroids. Studies performed with Mometasone cream 0.1% indicates that it is in the medium range of potency as compared with other topical corticosteroids. Corticosteroids have multiple mechanisms of action including anti-inflammatory activity, immunosuppressive properties, and anti-proliferative actions. Anti-inflammatory effects result from decreased formation, release and activity of the mediators of inflammation (eg. kinins, histamine, liposomal enzymes, prostaglandins, leukotrienes) which reduces the initial manifestations of the inflammatory process. Corticosteroids inhibit margination and subsequent cell migration to the area of injury, and also reverse the dilation and increased vessel permeability in the area, resulting in decreased access of cells to the sites of injury. This vasoconstrictive action decreases serum extravasation, swelling and discomfort. The immunosuppressive properties decrease the response to delayed and immediate hypersensitivity reactions (eg, type III and type IV). This results from inhibition of the toxic effect from antigen and antibody complexes that precipitate in vessel walls creating cutaneous allergic vasculitis, and by inhibiting the action of lymphokines, target cells, and macrophages which together produce allergic contact dermatitis reactions. Additionally, the access of sensitized T lymphocytes and macrophages to target cells may also be prevented by corticosteroids. The anti-proliferative effects reduce hyperplastic tissue characteristic of psoriasis.

Like other topical corticosteroids, mometasone furoate has anti-inflammatory, antipruritic, and vasoconstrictive properties. The mechanism of the anti-inflammatory activity of the topical steroids, in general, is unclear. However, corticosteroids are thought to act by the induction of phospholipase A₂ inhibitory proteins, collectively called lipocortins. Arachidonic acid is released from membrane phospholipids by phospholipase A₂. It is postulated that lipocortins control the biosynthesis of potent mediators of inflammation such as prostaglandins and leukotrienes by inhibiting the release of their common precursor arachidonic acid.

“Halobetasol”, a synthetic corticosteroid, structurally related to clobetasol belongs to category of high potency steroids. Halobetasol propionate has been shown to be a super high-potency topical corticosteroid by vasoconstrictor assay in solution, cream, and ointment formulations. Like other topical corticosteroids, halobetasol propionate has anti-inflammatory, antipruritic and vasoconstrictive actions. Corticosteroids are thought to act by the induction of phospholipase A2 inhibitory proteins, collectively called lipocortins. Human and animal studies indicate that less than 6% of the applied dose of halobetasol propionate enters the circulation within 96 hours following topical administration of the cream. Halobetasol being a steroid has multiple mechanisms of action including anti-inflammatory activity, immunosuppressive properties, and antiproliferative actions. Anti-inflammatory effects arise from decreased formation, release, and activity of the mediators of inflammation (eg, kinins, histamine, liposomal enzymes, prostaglandins, leukotrienes), which reduce the initial manifestations of the inflammatory process. Corticosteroids inhibit margination and subsequent cell migration to the area of injury, and also reverse the dilation and increased vessel permeability in the area, resulting in decreased access of cells to the sites of injury. This vasoconstrictive action decreases serum extravasation, swelling, and discomfort. The immunosuppressive properties decrease the response to delayed and immediate hypersensitivity reactions (eg, type III and type IV). This arises from inhibition of the toxic effect from antigen and antibody complexes that precipitate in vessel walls creating cutaneous allergic vasculitis, and by inhibiting the action of lymphokines, target cells, and macrophages that together produce allergic contact dermatitis reactions. Additionally, the access of sensitized T lymphocytes and macrophages to target cells may also be prevented by corticosteroids. The antiproliferative effects reduce hyperplastic tissue characteristic of psoriasis.

The topical formulations of the present invention include those suitable for topical, transdermal, rectal and buccal (e.g., sub-lingual) administration etc., preferably the formulations of the present invention are administered topically and are provided in the form of semisolid dosage forms. Suitable dosage forms include hydrous or anhydrous semisolids such as creams, ointments, gels, lotions or any other dosage form suitable for topical application and the like.

The term “hydrous” as used here in means the presence of water in a concentration range of about 5% to 95% in the composition.

The term “anhydrous” as used here in means the presence of water in a concentration range of less than 5% in the composition.

The topical formulations of the present invention with Mometasone and fusidic acid are useful in the treatment of infected eczemas such as secondarily infected dermatitis, including secondarily infected contact dermatitis, allergic contact dermatitis and atopic dermatitis with secondary bacterial infections of skin or in prevention of infection in cases of dermatitis, psoriasis, specially atopic dermatitis who are at risk of getting secondary bacterial infection or in prevention of infection in cases of dermatitis, specially atopic dermatitis who are at risk of getting secondary bacterial infection

The topical formulations of the present invention with Halobetasol and fusidic acid are useful in the treatment of infected steroid responsive dermatoses such as secondarily infected dermatoses including secondarily infected contact dermatitis, allergic contact dermatitis, atopic dermatitis, and other corticosteroid responsive dermatoses (CRD) with secondary bacterial infections of skin or in prevention of secondary bacterial infections in patients with non-infected dermatoses.

The topical formulations for the method of treatment of the present invention may be administered from 1 to 6 times daily and more usually from 2 to 4 times daily.

The topical pharmaceutical composition of the present invention may include suitable pharmaceutically acceptable carriers. A wide range of ingredients including, emollients; emulsifying agents; emulsion stabilizers and viscosity builders; humectants; odorants; preservatives, antioxidants, and chemical stabilizers; solvents; and thickening, stiffening, suspending agents; buffers, neutralizing agents and agents to adjust pH; coloring agents, opacifiers and decoloring agents, pigments; and antifoaming agents, skin feel modifiers and the like. Exemplary emollients include octyldodecanol, caprylic/capric triglycerides, castor oil, ceteareth-20, ceteareth-30, cetostearyl alcohol, ceteth 20, cetostearyl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, propylene glycol mono stearate squalane, steareth-2 or -100, stearic acid, stearyl alcohol, urea and the like. Exemplary emulsifying agents include Propylene glycol stearate, aluminum starch octenylsuccinate, ammonium hydroxide, amphoteric-9, beeswax, white beeswax, synthetic beeswax, carbomer 934, carbomer 934P, carbomer 940, ceteareth-20, ceteareth-30, cetearyl alcohol, ceteth 20, cetyl alcohol, cholesterol, cyclomethicone, diglycerides, dimethicone (e.g., dimethicone 350), disodium monooleamido sulfosuccinate, NF emulsifying wax, fatty acid pentaerythritol ester, glycerides, glyceryl monooleate, glyceryl monostearate, glyceryl stearate, lanolin, lanolin alcohol, hydrogenated lanolin, magnesium stearate, mineral oil, monoglycerides, polyethylene glycol, PEG stearate, polyethylene glycol 6000 distearate, polyethylene glycol 1000 monocetyl ether, polyethylene glycol monostearate, polyethylene glycol 400 monostearate, polyoxyethylene glycol fatty alcohol ethers, polyoxyl 20 cetostearyl ether, polyoxyl 40 stearate, polysorbate 20, polysorbate 40, polysorbate 60, polysorbate 80, polysorbates, PPG-26 oleate, propylene glycol stearate, quaternium-15, simethicone, sodium laureth sulfate, sodium lauryl sulfate, sorbitan esters, sorbitan monolaurate, sorbitan monooleate, sorbitan monopalmitate, sorbitan monostearate, sorbitan palmitate, sorbitan sesquioleate, steareth-2, steareth-100, stearic acid, stearyl alcohol, triethanolamine, trolamine and the like. Exemplary emulsion stabilizers and viscosity builders include carbomer 934, carbomer 934P, carbomer 940, cetearyl alcohol, cetostearyl alcohol, cetyl alcohol, cetyl stearyl alcohol, dextrin, diglycerides, disodium edetate, edetate disodium, glycerides, glyceryl monostearate, glyceryl stearate, hydroxypropyl cellulose, monoglycerides, plasticized hydrocarbon gel, polyethylene glycol 300, polyethylene glycol 400, polyethylene glycol 1450, polyethylene glycol 8000, polyethylene glycols, propylene glycol stearate, stearyl alcohol and the like. Exemplary humectants include glycerin, propylene glycol, sorbitol, urea and the like. Exemplary odorants include hypoallergenic perfume, menthol and the like. Exemplary preservatives, antioxidants, and chemical stabilizers include alcohol, benzyl alcohol, butylated hydroxyanisole, butylated hydroxytoluene, butylparaben, calcium acetate, caster oil, chlorocresol, Potassium sorbate, 4 chloro-m-cresol, citric acid, disodium edetate, Dowicil 200 (Dow), edetate disodium, ethoxylated alcohol, ethyl alcohol, glycerin, Glydant Plus (Lonza), 1,2,6-hexanetriol, Kathon CG (Rohm & Haas), Liquid Germall Plus (ISP Sutton Labs), Liquipar (ISP Sutton Labs), methylparaben, parabens, potassium sorbate, propyl gallate, propylene glycol, propylparaben, sodium bisulfite, sodium citrate, sodium metabisulfite, sorbic acid, tannic acid, triglycerides of saturated fatty acids, Ucarcide (Union Carbide), Vitamin E, zinc stearate and the like. Exemplary solvents include alcohol, castor oil, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palmitate, mineral oil, phosphoric acid, polyethylene glycol 300, polyethylene glycol 400, polyethylene glycol 1450, polyethylene glycol 8000, polyethylene glycol 1000 monocetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, octyldodecanol, propylene carbonate, propylene glycol, purified water, and SD alcohol 40, triglycerides of saturated fatty acids and the like. Exemplary thickening, stiffening and suspending agents include aluminum stearate, beeswax, white beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, white soft paraffin, petrolatum, white petrolatum polyethylene, propylene glycol stearate, starch, stearyl alcohol, wax, white wax, xanthan gum, bentonite and the like. Exemplary buffers, neutralizing agents and agents to adjust pH include phosphoric acid, ammonium hydroxide, citric acid, diisopropanolamine, hydrochloric acid, lactic acid, monobasic sodium phosphate, sodium citrate, sodium hydroxide, sodium phosphate, triethanolamine, trolamine and the like. Exemplary opacifiers/colorants include suitable for use may be organic and/or inorganic and the like. Suitable examples include titanium dioxide, and pre-dispersed titanium dioxide. Exemplary antifoaming agents include cyclomethicone, dimethicone (e.g., dimethicone 350), simethicone and the like. Exemplary skin feel modifiers Aluminum starch octenylsuccinate (gamma irradiated) and the like.

The invention is further exemplified with following examples and is not necessarily limited to the present formulations. It is obvious to those skilled in the art to find out the composition for other dosage forms and substitute the equivalent excipients as described in this specification or with the one known to the art.

### Example 1

### Mometasone Furoate and Fusidic Acid Topical Formulation

| Steps | Ingredients | % w/w |
|---|---|---|
| I | White Soft Paraffin | 63.869 |
| | White Bees Wax | 5.000 |
| | Promulgen G ™ | 7.000 |
| | Propylene Glycol Monostearate | 8.000 |
| | Aluminium Starch Octenylsuccinate (Dry | 5.000 |
| | Flow Pure ™) | |
| II | Purified water | 3.000 |
| | Hexylene Glycol | 6.000 |
| | Phosphoric Acid (10% w/v) | * |
| | Mometasone Furoate, Micronised | 0.100 |
| | Fusidic Acid (Micronised) | 2.000 |

* Quantity required to adjust pH of Hexylene Glycol & water solution up to pH 4.0

### Brief Manufacturing Process

1. Preparation of Oil Phase: White Soft Paraffin, White Bees Wax, Promulgen G™, Propylene Glycol Monostearate, Aluminium Starch Octenylsuccinate (Dry Flow Pure™) were heated to the temperature range 70-72° C.
2. Preparation of Drug Phase: pH of Hexylene Glycol & water solution was adjusted up to pH 4.0 using Phosphoric acid. This was heated up to 70° C. to 72° C. Mometasone Furoate and Fusidic Acid was added to this solution and dissolved with stirring.
3. Emulsification: Added drug phase to oily phase under stirring maintaining the temperature at 70° C. to 72° C., and then homogenized for 10 minutes. After homogenization, the mixed phase was slowly cooled to get a white to off white topical cream.

### Example 2

### Mometasone Furoate and Fusidic Acid Topical Formulation

| Step | Ingredients | % w/w |
|---|---|---|
| I | White Soft Paraffin | 6.000 |
| | Liquid Paraffin | 6.000 |
| | White Bees Wax | 1.500 |
| | Promulgen G | 4.000 |
| | Propylene Glycol Monostearate | 2.000 |
| | Polysorbate 60 | 1.500 |
| | Cetostearyl Alcohol | 6.000 |
| | Butylated Hydroxytoluene (BHT) | 0.050 |
| | Butylated Hydroxyanisole (BHA) | 0.050 |
| II | Purified water | 46.569 |
| III | Purified water | 1.000 |
| | Potassium Sorbate | 0.200 |
| IV | Phenoxyethanol | 0.500 |
| V | Purified water | 5.000 |
| | Polysorbate 60 | 0.500 |
| | Glycerin | 8.000 |
| | Fusidic Acid | 2.000 |
| VI | Purified Water | 3.000 |
| | Hexylene Glycol | 6.000 |
| | Phosphoric Acid solution (10%) | * |
| | Mometasone Furoate | 0.100 |

* Quantity required for adjusting pH of Hexylene Glycol & water solution up to pH 4.0 and then adjusting the final pH.

### Brief Manufacturing Process

1. Preparation of Oil Phase; White Soft Paraffin, Liquid Paraffin, White Bees Wax, Promulgen G™, Propylene Glycol Monostearate, Polysorbate 60, Cetostearyl Alcohol were heated to the temperature range 70-72° C. Butylated Hydroxytoluene and Butylated Hydroxyanisole were added.
2. Preparation of Aqueous Phase: Purified water was heated to the temperature range 70-72° C.
3. Emulsification Phase: Added oil phase to aqueous phase at 70° C. to 72° C. and homogenized for 15 minutes.
4. Mometasone Furoate Phase: Adjusted the pH of Hexylene Glycol & water solution up to pH 4.0 with phosphoric acid, followed by heating up to 70° C. to 72° C. Dissolved Mometasone Furoate under stirring. Add drug solution of Mometasone furoate to emulsification phase at 70° C.
5. Fusidic Acid Phase: Purified water & Glycerin were taken and Polysorbate 60 was added to it. Dispersed under stirring Fusidic Acid. Added Fusidic acid solution to emulsification phase at 70° C.
6. Added previously dissolved potassium sorbate solution in water to bulk at 40° C. under stirring.
7. Added Phenoxyethanol to the bulk at 40° C. under stirring. The bulk was then cooled under stirring slowly to get a cream.

### Physical Parameters Tested:

| | |
|---|---|
| Description | white coloured cream |
| pH | 5.0 to 5.5 |

### Example 3

### Mometasone Furoate and Fusidic Acid Topical Formulation

| Step | Ingredients | % w/w |
|---|---|---|
| I | White Soft Paraffin | 6.000 |
| | Light Liquid Paraffin | 6.000 |
| | Sorbitan Monosterate (SMS) | 2.000 |
| | Polyoxyl 20 Cetyl Ether (Brij 58) | 2.000 |
| | Cetostearyl alcohol | 8.000 |
| | White Bees Wax | 3.000 |
| | Butylated Hydroxyanisole (BHA) | 0.050 |
| | Butylated Hydroxytoluene (BHT) | 0.050 |
| II | Purified water | 52.069 |
| | Glycerin | 8.000 |
| III | Purified water | 1.000 |
| | Potassium Sorbate | 0.200 |
| IV | Phenoxyethanol | 0.500 |
| V | Purified water | 3.000 |
| | Hexylene Glycol | 6.000 |
| | Phosphoric Acid (10% w/v) | * |
| | Mometasone Furoate | 0.100 |
| | Fusidic Acid | 2.000 |

* Quantity required for adjusting pH of Hexylene Glycol & water solution up to pH 4.0 and then adjusting the final pH.

### Brief Manufacturing Process

1. Preparation of Oil Phase; White Soft Paraffin, Light Liquid Paraffin, Sorbitan Monostearate, Polyoxyl 20 Cetyl Ether, Cetostearyl Alcohol, White Bees Wax were heated to the temperature range 70-72° C. Butylated Hydroxytoluene and Butylated Hydroxyanisole were added.
2. Preparation of Aqueous Phase: Purified water and glycerine mixture was heated to a temperature range 70° C. to 72° C.
3. Emulsification Phase: Added oil phase to aqueous phase at 70° C. to 72° C. and homogenized for 15 minutes.
4. Preparation of Drug Phase: Adjusted the pH of Hexylene Glycol & water solution up to pH 4.0 with phosphoric acid. Followed by heating up to 70° C. to 72° C. Mometasone Furoate and Fusidic Acid were dissolved under stirring. Then added to emulsification phase at 70° C.
5. Added previously dissolved potassium sorbate solution in water to bulk at 40° C. under stirring.
6. Added Phenoxyethanol to the bulk at 40° C. under stirring. The bulk was then cooled under stirring slowly to get a cream.

### Physical Parameters:

| | |
|---|---|
| Description | white coloured cream |
| pH | 5.0 to 5.5 |

### Example 4

### Halobetasol Propionate and Fusidic Acid Topical Formulation

| Step | Ingredients | % w/w |
|---|---|---|
| I | White Soft Paraffin | 4.000 |
| | Polyoxyl 20 Cetyl Ether (Brij 58) | 1.800 |
| | Cetyl Alcohol | 2.000 |
| | Cetostearyl alcohol | 8.000 |
| | Dimethicone 350 | 0.500 |
| | Isopropyl Palmitate | 5.500 |
| | Butylated Hydroxytoluene (BHT) | 0.100 |
| | Phenoxyethanol | 0.700 |
| II | Purified water | 58.626 |
| | Potassium Sorbate | 0.200 |
| III | Glycerin | 2.000 |
| | Polyoxyl 20 Cetyl Ether (Brij 58) | 0.050 |
| | Water | 0.750 |
| | Halobetasol Propionate | 0.050 |
| IV | Glycerin | 6.000 |
| | Polyoxyl 20 Cetyl Ether (Brij 58) | 0.200 |
| | Purified Water | 7.000 |
| | Fusidic Acid | 2.000 |
| V | Phosphoric Acid solution 10% | 0.524 |

### Brief Manufacturing Process

1. Preparation of Oil Phase; White Soft Paraffin, part of Polyoxyl 20 Cetyl Ether, Cetyl alcohol, Cetostearyl Alcohol, Dimethicone 350, Isopropyl Palmitate, Butylated Hydroxytoluene and Phenoxyethanol were heated to the temperature range 70-72° C.
2. Preparation of Aqueous phase: Dissolved potassium sorbate in water and heated to a temperature range 70-72° C.
3. Emulsification: Added oil phase to aqueous phase at 70° C. to 72° C. and homogenized for 15 minutes, followed by cooling.
4. Heated glycerin up to 60° C. then added Polyoxyl 20 Cetyl Ether (Brij 58) to melt completely and added water to maintain temperature up to 40° to 42° C. At 42° C. added under stirring Halobetasol Propionate to disperse completely.
5. Heated glycerin up to 60° C. then added Polyoxyl 20 Cetyl Ether (Brij 58) to melt completely and added water to maintain temperature up to 40° to 42° C. At 42° C. added under stirring Fusidic Acid to disperse completely.
6. Added Halobetasol solution to the bulk at 50° C. under stirring.
7. Added Fusidic Acid solution to the bulk at 40° C. under stirring.
8. Added Phosphoric Acid solution 10% to the bulk at 40° C. under stirring. The bulk was then cooled under stirring slowly to get a cream.

### Physical Parameters:

| Description | white coloured cream |
|---|---|
| pH at 25° C. | 4.5-6.00 |

### Example 5

### Halobetasol Propionate and Fusidic Acid Topical Formulation

| Step | Ingredients | % w/w |
|---|---|---|
| I | White Soft Paraffin | 4.000 |
| | Polyoxyl 20 Cetyl Ether (Brij 58) | 1.800 |
| | Cetyl Alcohol | 2.000 |
| | Cetostearyl alcohol | 8.000 |
| | Dimethicone 350 | 0.500 |
| | Isopropyl Palmitate | 5.500 |
| | Butylated Hydroxytoluene (BHT) | 0.100 |
| | Benzyl Alcohol | 1.000 |
| II | Purified water | 63.320 |
| | Potassium Sorbate | 0.200 |
| III | Glycerin | 2.000 |
| | Polyoxyl 20 Cetyl Ether (Brij 58) | 0.050 |
| | Water | 0.750 |
| | Halobetasol Propionate (Micronized) | 0.050 |
| IV | Glycerin | 6.000 |
| | Polyoxyl 20 Cetyl Ether (Brij 58) | 0.200 |
| | Purified Water | 2.000 |
| | Fusidic Acid(Micronized) | 2.000 |
| V | Phosphoric Acid solution (10%) | 0.530 |

### Brief Manufacturing Process

1. Preparation of Oil Phase; White Soft Paraffin, part of Polyoxyl 20 Cetyl Ether, Cetyl alcohol, Cetostearyl Alcohol, Dimethicone 350, Isopropyl Palmitate, Benzyl alcohol were heated to the temperature range 70-72° C.
2. Preparation of Aqueous phase: Dissolved potassium sorbate in water and heated to a temperature range 70-72° C.
3. Emulsification: Added oil phase to aqueous phase at 70° C. to 72° C. and homogenized for 15 minutes, followed by cooling.
4. Heated glycerin up to 60° C. then added Polyoxyl 20 Cetyl Ether (Brij 58) to melt completely and added water to maintain temperature up to 40° to 42° C. At 42° C. added under stirring Halobetasol Propionate to disperse completely.
5. Heated glycerin up to 60° C. then added Polyoxyl 20 Cetyl Ether (Brij 58) to melt completely and added water to maintain temperature up to 40° to 42° C. At 42° C. added under stirring Fusidic Acid to disperse completely.
6. Added Halobetasol solution to the bulk at 50° C. under stirring.
7. Added Fusidic Acid solution to the bulk at 40° C. under stirring.
8. Added Phosphoric Acid solution 10% to the bulk at 40° C. under stirring. The bulk was then cooled under stirring slowly to get a cream.

### Example 6

### Halobetasol Propionate and Fusidic Acid Topical Formulation

| Step | Ingredients | % w/w |
|---|---|---|
| I | White Soft Paraffin | 4.000 |
| | Polyoxyl 20 Cetyl Ether (Brij 58) | 2.000 |
| | Cetyl Alcohol | 2.000 |
| | Cetostearyl alcohol | 8.000 |
| | Dimethicone 350 | 0.500 |
| | Isopropyl Palmitate | 6.000 |
| | Butylated Hydroxytoluene (BHT) | 0.100 |
| | Benzyl Alcohol | 1.000 |
| II | Purified water | 63.350 |
| III | Diethylene Glycol Monoethyl Ether (Transcutol | 1.000 |
| | P) | |
| | Halobetasol Propionate | 0.050 |
| IV | Hexylene Glycol | 5.000 |
| | Glycerin | 4.000 |
| | Polysorbate 60 (Tween 60) | 1.000 |
| | Fusidic Acid | 2.000 |

### Brief Manufacturing Process

1. Preparation of Oil Phase; White Soft Paraffin, Polyoxyl 20 Cetyl Ether, Cetyl alcohol, Cetostearyl Alcohol, Dimethicone 350, Isopropyl Palmitate, BHT, Benzyl alcohol were heated to the temperature range 70-72° C.
2. Preparation of Aqueous phase: Heated water to a temperature range 70-72° C.
3. Emulsification: Added oil phase to aqueous phase at 70° C. to 72° C. and homogenized for 15 minutes, followed by cooling.
4. In Separate container Diethylene Glycol Monoethyl Ether (Transcutol P) was taken, to this added under stirring Halobetasol Propionate. Continued stirring for 5 minutes to dissolve completely and added to the bulk at 50° C. under stirring.
5. Heated Hexylene Glycol up to 50° C. then added glycerin and Polysorbate 60 (Tween 60) and maintained temperature up to 50°-52° C. At 52° C. added under stirring Fusidic Acid to dissolve completely. Added the Fusidic Acid solution to the main bulk at 40° C. under stirring. The bulk was then cooled under stirring slowly to get a cream.

### Example 7

### Halobetasol Propionate and Fusidic Acid Topical Formulation

| Step | Ingredients | % w/w |
|---|---|---|
| I | White Soft Paraffin | 9.000 |
| | Cetostearyl Alcohol | 10.000 |
| | liquid Paraffin | 7.000 |
| | Polysorbate 60 (Tween 60) | 2.000 |
| | Vitamin E (As dl-alpha Tocopherol | 0.050 |
| | Acetate) | |
| | Butylated Hydroxytoluene | 0.050 |
| II | Purified Water | 57.580 |
| | Potassium sorbate | 0.270 |
| III | Diethylene Glycol Monoethyl Ether | 1.000 |
| | (Transcutol P) | |
| | Halobetasol Propionate | 0.050 |
| IV | Glycerin | 10.000 |
| | Polysorbate 60 (Tween 60) | 1.000 |
| | Fusidic Acid | 2.000 |

### Brief Manufacturing Process

1. Preparation of Oil Phase; White Soft Paraffin, Cetostearyl Alcohol, Liquid paraffin, Polysorbate 60, Vitamin E, Butylated Hydroxytoluene were heated to the temperature range 70-72° C.
2. Preparation of Aqueous phase: Potassium sorbate dissolved in water and heated to a temperature range 70-72° C.
3. Emulsification: Added oil phase to aqueous phase at 70° C. to 72° C. and homogenized for 15 minutes, followed by cooling.
4. In Separate container Diethylene Glycol Monoethyl Ether (Transcutol P) was taken, to this added under stirring Halobetasol Propionate. Continued stirring for 5 minutes to dissolve completely and added to the bulk at 50° C. under stirring.
5. Heated glycerin and Polysorbate 60 (Tween 60) up to 50° C. then added Fusidic Acid to disperse completely. Added the Fusidic Acid dispersion to the main bulk at 40° C. under stirring. The bulk was then cooled under stirring slowly to get a cream.

### Example 8

### Halobetasol Propionate and Fusidic Acid Topical Formulation

| Step | Ingredients | % w/w |
|---|---|---|
| I | Cetyl Alcohol | 12.000 |
| | Isopropyl Isostearate (Prisorin 2021) | 3.000 |
| | Isopropyl Palmitate | 2.000 |
| | Polyoxyl 20 Cetyl Ether (Brij 58) | 2.000 |
| | Polyoxyethylene (21) Stearyl Ether (Brij | 3.000 |
| | 721) | |
| II | Purified Water | 56.663 |
| III | Purified Water | 5.000 |
| | Imiduria | 0.200 |
| IV | Purified Water | 3.000 |
| | Methylisothiazolinone & | 0.050 |
| | Methylchloroisothiazolinone (Kathon CG) | |
| V | Glycerin | 8.000 |
| | Polyoxyl 20 Cetyl Ether (Brij 58) | 1.000 |
| | Fusidic Acid (Micronized) | 2.037 |
| VI | Glycerin | 2.000 |
| | Halobetasol Propionate (Micronized) | 0.050 |
| | Total | 100.000 |

1. Preparation of Oil Phase: Heated Cetostearyl Alcohol, Isopropyl Isostearate, Polyoxyl 20 Cetyl Ether (Brij 58), Polyoxyethylene (21) Stearyl Ether (Brij 721) were heated to the temperature range 70-72° C.
2. Preparation of Aqueous phase: Heated part of water to a temperature range 70-72° C.
3. Emulsification: Added oil phase to aqueous phase at 70° C. to 72° C. and homogenized for 15 minutes, followed by cooling.
4. Added Imiduria solution in water to the bulk at 40°-42° C. under stirring.
5. Added Kathon CG dispersion to the bulk at 40°-42° C. under stirring.
6. Heated glycerin and Polyoxyl 20 Cetyl Ether (Brij 58) up to 50° C. then added Fusidic Acid to disperse completely. Added Fusidic Acid dispersion to the main bulk at 40° C. under stirring.
7. In Separate container, Glycerin was taken, to this added under stirring Halobetasol Propionate. Continued stirring for 5 minutes to disperse completely and added to the bulk at 40° C. under stirring. The bulk was then cooled under stirring slowly to get a cream.

The compositions of the present invention, example 1 and 4 were tested for clinical efficacy on human volunteers for indications described herein. The formulation of the present invention were assessed for efficacy, safety and tolerability of combination of Mometasone 0.1% w/w plus Fusidic acid 2% w/w cream, Example 1 vs Mometasone 0.1% w/w cream for prevention of secondary bacterial infections in patients with corticosteroid-responsive dermatoses. The combination preparation of Momometasone and Fusidic acid of the present invention was compared with marketed preparation Momate® containing 0.1% w/w Momatasone furoate, available in India, Malaysia and Philippines, marketed by Glenmark Pharmaceutical Ltd.

Prospective, randomized, multicentre, open label study was conducted with 50 Male & female (post-menopausal, surgically sterilized or practicing a reliable method of birth control) patients with age ranging from 12 to 65 years. The duration of the study was 3 weeks, including a 2-week active treatment period, preceded by a 1-week washout phase. The patients selected were clinically diagnosed for corticosteroid-responsive dermatoses without secondary bacterial infections. The exclusion criteria for the patients were pregnant and lactating women, Serious skin disorders, dyspigmentation and extensive scarring in the affected areas. Hypersensitivity to Mometasone or Fusidic acid or cream base. Immuno-compromised states and patients with systemic infections. Patients who have participated in a new drug study in the past 6 months. Patients with severe cardiac, hepatic, renal, or cerebrovascular disease, malignancy, chronic uncontrolled systemic diseases e.g., diabetes, hypertension, asthma, collagen disorders, etc. or any other serious medical illness.

Study Plan

| | Screening | Visit I | | |
|---|---|---|---|---|
| | Visit | (Baseline/ | Visit II | Visit III |
| Activity/Observation | (Day −7) | Day 0) | (Day 6-7) | (Day 13-14) |
| Demographic | X | | | |
| Variables | | | | |
| History and Physical | X | | | |
| Examination | | | | |
| Inclusion/Exclusion | X | | | |
| Criteria | | | | |
| Informed Consent | X | | | |
| Clinical Evaluation | X | X | X | X |
| Laboratory and | X | | | |
| other investigations | | | | |
| Study drug treatment | | X | X | X |
| Adverse Events | | X | X | X |
| Patient Compliance | | | X | X |
| Overall Assessment of | | | X | X |
| Efficacy | | | | |
| Bacteriological | X | | | X |
| evaluation | | | | |

### Screening Visit:

Demographic data and history of the patients were recorded and a brief physical examination was performed. Patients were recruited based on the inclusion and exclusion criteria. Written, informed consent was obtained from these patients. Clinical assessment of the condition under study, laboratory investigations, chest x-ray & ECG were carried out at this visit. Patients were given a 1-week washout period following which study treatment was initiated.

### Clinical Efficacy Parameters Assessed:

The study was designed to monitor clinical signs and symptoms such as Scaling, Erythema, Itching, Induration/Edema, Pain, Exudation/Crusting and Total sign/symptom. In addition Severity of lesion, Discharge for lesion, Bacteriological findings and Bacteriological findings including Staphylococcus aureus, Staphylococcus epidermidis, Streptococcus spp, Enterobacteriaceae were also monitored.

### Study Treatment

Patients received Mometasone 0.1% plus Fusidic acid 2% cream to be applied over the affected area/s twice daily as a thin film and rubbed in gently and completely, with each application separated by about 12 hours.

### Overall Global Assessment of Efficacy

At the end of study, an overall assessment of the efficacy of the medication was performed by both the investigator and the patient.

Physician's Global Evaluation included the following parameters:

| Grade | Evaluation |
|---|---|
| 1 | Complete resolution (100% remission of signs |
| | and symptoms) |
| 2 | Excellent (90-99% improvement) |
| 3 | Marked (75-89% improvement) |
| 4 | Moderate (50-74% improvement) |
| 5 | Slight (30-49% improvement) |
| 6 | No appreciable improvement (0-29%) |
| 7 | Worse (<0%) |

Patient's Global Evaluation

| Grade | Evaluation |
|---|---|
| 1 | Greatly improved |
| 2 | Somewhat improved |
| 3 | Same |
| 4 | Somewhat worse |
| 5 | Much worse |

Patient Compliance was assessed on visits II, III and IV by direct questioning to the patients.

### Results of the Study:

The study data for assessment for efficacy of combination of Mometasone 0.1% w/w and Fusidic acid 2% w/w cream (Example 1 of the present invention) vs Mometasone 0.1% w/w cream, Momate® for prevention of secondary bacterial infections in patients with corticosteroid-responsive dermatoses were pooled and results were analyzed using a non-parametric test. All tests were two tailed & p<0.05 were considered to be significant.

TABLE 1
Table 1: Results of Clinical symptoms assessment with combination of
Mometasone 0.1% w/w and Fusidic acid 2% w/w cream (Example 1 of the present
invention) vs Mometasone 0.1% w/w cream, Momate ® for prevention of
secondary bacterial infections in patients with corticosteroid-responsive dermatoses.

| | Mean Total Score ( X̅ ± SD) | | | | | |
|---|---|---|---|---|---|---|
| | Indication | | | | | |
| Duration | Effect on Scaling | | Effect on Erythema | | Effect on Itching | |
| in days | Example 1 | Momate ® | Example 1 | Momate ® | Example 1 | Momate ® |
| Baseline | 2.52 ± 0.95 | 2.60 ± 0.91 | 2.46 ± 0.88 | 2.35 ± 0.73 | 2.39 ± 0.80 | 2.44 ± 0.93 |
| 6-7 | *1.72 ± 0.69 | *1.95 ± 0.77 | *1.23 ± 0.72 | @*1.64 ± 0.90 | *1.28 ± 0.67 | @*1.78 ± 0.59 |
| 13-14 | *0.54 ± 0.55 | @*1.01 ± 0.63 | *0.40 ± 0.49 | @*0.98 ± 0.73 | *0.59 ± 0.63 | @*1.04 ± 0.65 |
| | Indication | | | | | |
| | Effect on | | | | | |
| Duration | Induration/Edema | | Effect on Pain | | Effect on Exudation | |
| in days | Example 1 | Momate ® | Example 1 | Momate ® | Example 1 | Momate ® |
| Baseline | 2.01 ± 0.76 | 1.98 ± 0.85 | 2.27 ± 0.90 | 2.19 ± 0.78 | 1.72 ± 0.78 | 1.65 ± 0.59 |
| 6-7 | *0.98 ± 0.52 | @*1.30 ± 0.57 | *1.09 ± 0.71 | @*1.59 ± 0.66 | *0.80 ± 0.68 | @*1.08 ± 0.79 |
| 13-14 | *0.39 ± 0.45 | @*0.94 ± 0.77 | *0.40 ± 0.59 | @*0.85 ± 0.71 | *0.41 ± 0.49 | @*0.72 ± 0.66 |

By Wilcoxon Sign Rank Test *P < 0.05 Vs Basal, Significant
By Mann Whitney U Test @Between Groups P < 0.05 Significant

Table 1 shows that mean score of scaling were 2.52 and 2.60 respectively in patients treated with composition as described in Example 1 of the present invention and patients treated with Momate® at baseline which was same and difference was not statistically significant. After the end of treatment, mean score had a fall of 78.6% among Example 1 group, which was significantly more as compared to 61.2% in Momate® group. After the end of treatment, mean score for Erythema had a fall of 83.7% among patients treated with Example 1 of the present invention which was significantly more as compared to 58.3% in Momate® group. After the end of treatment, mean score for itching had a fall of 75.3% among Example 1 group which was significantly more as compared to 57.4% in Momate® group. After the end of treatment for Induration/edema, mean score had a fall of 80.6% among Example 1 group which was significantly more as compared to 52.5% in Momate® group. After the end of treatment, mean score for pain had a fall of 82.4% among Example 1 group which was significantly more as compared to 61.2% in Momate® group. After the end of treatment, mean score for exudation had a fall of 76.2% among Example 1 group which was significantly more as compared to 56.4% in Momate® group. After the end of treatment, mean score for total signs/symptoms had a fall of 79.6% among Example 1 group which was significantly more as compared to 58.1% in Momate® group.

TABLE 2
Table 2: Results of changes in severity of lesions with combination of
Mometasone 0.1% w/w and Fusidic acid 2% w/w cream (Example 1 of the present
invention) vs Mometasone 0.1% w/w cream, Momate ® for prevention of
secondary bacterial infections in patients with corticosteroid-responsive dermatoses.

| | Changes In Severity Of Lesions Between Two Groups | | | | | |
|---|---|---|---|---|---|---|
| | Example 1 | | | Momate ® | | |
| | Baseline | Day 6-7 | Day 13-14 | Baseline | Day 6-7 | Day 13-14 |
| | (N = 25) | (N = 25) | (N = 25) | (N = 26) | (N = 26) | (N = 26) |
| Severity | No. % | No. % | No. % | No. % | No. % | No. % |
| Mild | 05 (20.0) | 08 (32.0) | *22 (88.0) | 06 (23.1) | 07 (26.9) | 12 (46.2) |
| Moderate | 10 (40.0) | 12 (48.0) | 02 (08.0) | 09 (34.6) | 11 (42.3) | 09 (34.6) |
| Severe | 10 (40.0) | 05 (20.0) | 01 (04.0) | 11 (42.3) | 08 (30.8) | 05 (19.2) |

By Chi - Square test *P < 0.05 Significant

72.1-80.0% of the total study cases had a mild to moderate severity of lesions in both the groups at baseline. At the end of treatment 88.0% of total cases had mild severity of lesion in Example 1 group which was significantly low as compared to 46.2% among Momate® group.

TABLE 3
Table 3: Results of changes in discharge of lesions with combination of
Mometasone 0.1% w/w and Fusidic acid 2% w/w cream(Example 1 of the present
invention) vs Mometasone 0.1% w/w cream, Momate ® for prevention of
secondary bacterial infections in patients with corticosteroid-responsive dermatoses

| | Changes In Discharge For Lesions Between Two Groups | | | | | |
|---|---|---|---|---|---|---|
| | Example 1 | | | Momate ® | | |
| | | Day | Day | | Day | Day |
| | Baseline | 6-7 | 13-14 | Baseline | 6-7 | 13-14 |
| Type of | (N = 25) | (N = 25) | (N = 25) | (N = 26) | (N = 26) | (N = 26) |
| Discharge | No. % | No. % | No. % | No. % | No. % | No. % |
| Absent | — (—) | *07 (28.0) | 16 (64.0) | — (—) | 03 (11.5) | 06 (23.1) |
| Serous | 04 (16.0) | *10 (40.0) | 06 (24.0) | 05 (19.2) | 06 (23.1) | 09 (34.6) |
| Purulent | 14 (56.0) | 05 (20.0) | *02 (08.0) | 15 (57.7) | 12 (46.2) | 07 (26.9) |
| Mucopurulent | 07 (28.0) | 03 (12.0) | *01 (04.0) | 06 (23.1) | 05 (19.2) | 04 (15.4) |

By Chi - Square test *P < 0.05 Significant

In this study group 80.8-84.0% of the total study cases had a purulent to mucopurulent type of discharge for lesion in both the groups at baseline. After treatment at the end of 6-7 days, 68.0% of the total cases had a serous to no discharges in Example 1 group which was significantly more as compared to 34.6% among Momate® group. At the end of treatment 12.0% of total cases had purulent to mucopurulent type of discharge in Example 1 group which was significantly low as compared to 42.3% among Momate® group. The bacteriological findings in the study indicated that 80.8-88.0% of the total cases had a significant bacteriological growth in both the groups at baseline. The common bacteriological isolates were staphylococcus aureus, streptococcus spp followed by epidermidis and proteus spp in both the groups. After the treatment only 16.0% of the cases had a significant growth among the patients treated with Example 1 of the present invention which was significantly very low as compared to 57.5% in patients treated with Momate® cream. As per Global assessment of efficacy of treatment by Physicians, 76.0% of total cases showed complete resolution to 90-99% resolution among total sign & symptoms in Example 1 group which was significantly more as compared to 23.1% among Momate® group. As per Global assessment of efficacy of treatment by patients, 80.0% of total cases had greatly improvement in Example 1 group which was significantly more as compared to 23.1% among Momate® group.

The formulation of the present invention were also assessed for efficacy, safety and tolerability of combination of mometasone 0.1% w/w and fusidic acid 2% w/w cream, Example 1 in treatment of patients of corticosteroid-responsive dermatoses with secondary bacterial infections. Prospective, randomized, monocentre, open label study was conducted in one centre with 25 Male & female (post-menopausal, surgically sterilized or practicing a reliable method of birth control) patients with age ranging from 12 to 65 years. The duration of the study was 3 weeks, including a 2-week active treatment period, preceded by a 1-week washout phase. The efficacy variables included changes in mean scores of scaling, erythema, pruritus/itching, induration/edema, pain, exudation/crusting, total sign/symptom score, severity of lesion, discharge from lesions and overall global assessment of efficacy by physician/patients and monitoring of treatment-emergent adverse events. The study data was pooled and results were analyzed using a non-parametric test. All tests were two tailed & p<0.05 were considered to be significant.

TABLE 4
Table 4: Results of clinical symptoms with combination of Mometasone 0.1%
w/w and Fusidic acid 2% w/w cream (Example 1 of the present invention) in
treatment of patients of corticosteroid-responsive dermatoses with secondary
bacterial infections.

| | Mean Total Score ( X̅ ± SD) | | | | | |
|---|---|---|---|---|---|---|
| | Indication | | | | | |
| Duration | Effect on Scaling | | Effect on Erythema | | Effect on Itching | |
| in days | Example 1 | % change | Example 1 | % change | Example 1 | % change |
| Baseline | 2.48 ± 0.89 | — | 2.36 ± 0.75 | — | 2.40 ± 0.61 | — |
| 6-7 | *1.86 ± 0.55 | 25.0 | *1.27 ± 0.89 | 46.2 | *1.32 ± 0.81 | 45.0 |
| 13-14 | *0.52 ± 0.61 | 79.0 | *0.36 ± 0.48 | 84.7 | *0.67 ± 0.52 | 72.1 |
| | Indication | | | | | |
| | Effect on | | | | | |
| Duration | Induration/Edema | | Effect on Pain | | Effect on Exudation | |
| in days | Example 1 | % change | Example 1 | % change | Example 1 | % change |
| Baseline | 1.95 ± 0.62 | — | 2.11 ± 0.63 | — | 1.67 ± 0.72 | — |
| 6-7 | *1.00 ± 0.49 | 48.7 | *1.17 ± 0.55 | 44.5 | *0.84 ± 0.65 | 49.7 |
| 13-14 | *0.42 ± 0.67 | 78.5 | *0.32 ± 0.49 | 84.8 | *0.32 ± 0.51 | 80.8 |

By Wilcoxon Sign Rank Test *P < 0.05 Vs Basal, Significant

In this study group mean total score of symptoms were 12.97 at baseline. After the treatment at the end of 6-7 days mean score had a significant reduction i.e. 42.5%. At the end of 13-14 days mean score had a fall of 82.7% from baseline.

Results showed that there was a significant decrease (p<0.05) in the mean symptom/sign scores as well as the total scores 1st week onwards and was significant sustained till two weeks (p<0.05).

TABLE 5
Results of changes in severity of lesions with combination of
Mometasone 0.1% w/w and Fusidic acid 2% w/w cream (Example 1
of the present invention) in treatment of patients of corticosteroid-
responsive dermatoses with secondary bacterial infections
Table 5: Results of changes in severity of lesions

| | | |
|---|---|---|
| Baseline | Day 6-7 | Day 13-14 |
| (N = 25) | (N = 25) | (N = 25) |

| Severity | No. | % | No. | % | No. | % |
|---|---|---|---|---|---|---|
| Mild | 04 | 16.0 | *07 | 28.0 | *20 | 80.0 |
| Moderate | 09 | 36.0 | *14 | 56.0 | 03 | 12.0 |
| Severe | 12 | 48.0 | 04 | 16.0 | 02 | 08.0 |

By Chi - Square Test *P < 0.05 Vs Baseline, Significant

Table 5 shows that 52.0% of the total study cases had a mild to moderate severity of lesions at baseline. After treatment at the end of 6-7 days, 84.0% of the total cases showed mild to moderate severity of lesions which was considerable change and difference was significant. At the end of treatment 80.0% of the cases had a mild severity of lesions which was significant change from baseline.

TABLE 6
Results of changes in discharge of lesions with combination of
Mometasone 0.1% w/w and Fusidic acid 2% w/w cream (Example 1
of the present invention) in treatment of patients of corticosteroid-
responsive dermatoses with secondary bacterial infections
Table 6: Results of changes in discharge of lesions

| | Day 3 | | Day 6-7 | | Day 13-14 | |
|---|---|---|---|---|---|---|
| | (N = 25) | | (N = 25) | | (N = 25) | |
| Type of Discharge | No. | % | No. | % | No. | % |
| Absent | — | — | 06 | 24.0 | *17 | 68.0 |
| Serous | 05 | 20.0 | 11 | 44.0 | 04 | 16.0 |
| Purulent | 14 | 56.0 | *06 | 24.0 | 03 | 12.0 |
| Mucopurulent | 06 | 24.0 | *02 | 08.0 | 01 | 04.0 |

By Chi - Square Test *P < 0.05 Vs Baseline, Significant

Table 6 indicates 80.0% of the total study cases had a purulent to mucopurulent type of discharge for lesions at baseline. After treatment at the end of 6-7 days, only 32.00% of the total cases showed same type of discharge for lesions which was statistically significant from baseline. At the end of treatment 68.0% of the cases did not had a discharge and 16.0% serous discharge. 88.0% of the total cases had a bacteriological growth at baseline. Out of which 56.0% had a Staphylococcus aureus, 20.0% Streptococcus Spp and 8.0% Staphylococcus. epidermidis. At the end of treatment only 8.0% of the cases had a growth. There was a significant decrease (92%) in bacteriological growth compared to baseline (P<0.05). According to the physician's assessment, 32.0% of the total study cases had a 100% remission of sign and symptoms, 44.0% showed a 90-99% improvement followed by 20.0% cases showed a moderate improvement. According to patients assessment, 84.0% of the total study cases showed a greatly improvement and 16.0% had a somewhat improvement after the treatment.

Treatment with combination of Mometasone furoate 0.1% w/w and Fusidic acid 2% w/w cream, Example 1 of the present invention, proved to be effective in 100% cases with 96% cases showing marked improvement to complete resolution of symptoms, the remaining 4% cases also showed moderate improvement. These results are interpreted to be superior to the results obtained by Javier P R et al 1986 (Ref: Javier P R, Ortiz M, Torralba L, Montinola F L, Ke M L, Canete R. Fusidic acid/betamethasone in infected dermatoses—a double-blind comparison with neomycin/betamethasone. Br J Clin Pract. 1986; 40:235-8), with the combination of betamethasone 0.1% plus fusidic acid 2% cream, where efficacy was seen in 85% cases. The combination of Mometasone furoate 0.1% w/w and Fusidic acid 2% w/w cream, Example 1 of the present invention, also proved to be effective in eradication of the bacterial organisms in 92% of cases. This was found to be superior to the results obtained by Hjorth N, et al 1985 (Ref: Hjorth N, Schmidt H, Thomsen K. Fusidic acid plus betamethasone in infected or potentially infected eczema. Pharmatherapeutica. 1985; 4:126-31.), where bacteriological cures were 67% and by Javier P R et al 1986, where bacteriological cures were 78%. Therapy with the combination of Mometasone furoate 0.1% w/w and Fusidic acid 2% w/w cream of the present invention provided a fast, better efficacy and safe tolerability.

The formulation of the invention were assessed for efficacy, safety and tolerability of combination of Halobetasol propionate 0.05% w/w and Fusidic acid 2% w/w cream, Example 4 vs Halobetasol propionate 0.05% w/w cream, Halbate®, marketed by Glenmark Pharmaceutical Ltd. in India, for prevention of secondary bacterial infections in patients with non-infected dermatoses.

Prospective, randomized, multicentre, open label study was conducted with 25 Male & female (post-menopausal, surgically sterilized or practicing a reliable method of birth control) patients with age ranging from 12 to 65 years. The duration of the study was 3 weeks, including a 2-week active treatment period, preceded by a 1-week washout phase. The patients selected were clinically diagnosed for non-infected dermatoses. The exclusion criteria for the patients were pregnant and lactating women, Serious skin disorders, dyspigmentation and extensive scarring in the affected areas. Hypersensitivity to halobetasol or Clobetasol or salicylic acid or ointment base. Immuno-compromised states and patients with systemic infections. Patients who have participated in a new drug study in the past 6 months. Patients with severe cardiac, hepatic, renal, or cerebrovascular disease, malignancy, chronic uncontrolled systemic diseases e.g., diabetes, hypertension, asthma, collagen disorders, etc. or any other serious medical illness.

Study Plan

| | Screening | Visit I | | |
|---|---|---|---|---|
| | Visit | (Baseline/ | Visit II | Visit III |
| Activity/Observation | (Day −7) | Day 0) | (Day 4-6) | (Day 8-10) |
| Demographic | X | | | |
| Variables | | | | |
| History and Physical | X | | | |
| Examination | | | | |
| Inclusion/Exclusion | X | | | |
| Criteria | | | | |
| Informed Consent | X | | | |
| Clinical Evaluation | X | X | X | X |
| Laboratory and | X | | | |
| other investigations | | | | |
| Study drug treatment | | X | X | X |
| Adverse Events | | X | X | X |
| Patient Compliance | | | X | X |
| Overall Assessment of | | | X | X |
| Efficacy | | | | |
| Bacteriological | X | | | X |
| evaluation | | | | |

### Screening Visit:

Demographic data and history of the patients were recorded and a brief physical examination was performed. Patients were recruited based on the inclusion and exclusion criteria. Written, informed consent was obtained from these patients. Clinical assessment of the condition under study, laboratory investigations, chest x-ray & ECG were carried out at this visit. Patients were given a 1-week washout period following which study treatment was initiated.

### Clinical Efficacy Parameters Assessed:

The study was designed to monitor Clinical signs and symptoms such as Scaling, Erythema, Itching, Induration/Edema, Pain, Exudation/Crusting and Total sign/symptom. In addition Severity of lesion, Discharge for lesion, Bacteriological findings and Bacteriological findings including Staphylococcus aureus, Staphylococcus epidermidis, Streptococcus spp, Enterobacteriaceae were also monitored.

### Study Treatment

Patients received combination of Halobetasol propionate 0.05% w/w and Fusidic acid 2% w/w cream or Halobetasol propionate 0.05% w/w cream according to randomization to be applied over the affected area/s twice daily as a thin film and rubbed in gently and completely, with each application separated by about 12 hours.

### Overall Global Assessment of Efficacy

At the end of study, an overall assessment of the efficacy of the medication was performed by both the investigator and the patient.

Physician's Global Evaluation included the following parameters:

| Grade | Evaluation |
|---|---|
| 1 | Complete resolution (100% remission of signs |
| | and symptoms) |
| 2 | Excellent (90-99% improvement) |
| 3 | Marked (75-89% improvement) |
| 4 | Moderate (50-74% improvement) |
| 5 | Slight (30-49% improvement) |
| 6 | No appreciable improvement (0-29%) |
| 7 | Worse (<0%) |

Patient's Global Evaluation

| Grade | Evaluation |
|---|---|
| 1 | Greatly improved |
| 2 | Somewhat improved |
| 3 | Same |
| 4 | Somewhat worse |
| 5 | Much worse |

Patient Compliance was assessed on visits II, III and IV by direct questioning to the patients.

### Results of the Study:

The study data for assessment for efficacy of combination of Halobetasol propionate 0.05% w/w and Fusidic acid 2% w/w cream, Example 4 of the present invention vs Halobetasol propionate 0.05% w/w cream, Halbate® in prevention of secondary bacterial infections in patients with non-infected dermatoses were pooled and results were analyzed using a non-parametric test. All tests were two tailed & p<0.05 were considered to be significant.

TABLE 1A
Table 1A: Results of Clinical symptoms assessment with combination of
Halobetasol propionate 0.05% plus Fusidic acid 2% cream, Example 4 of the
present invention vs Halobetasol propionate 0.05% cream, Halbate ® in prevention
of secondary bacterial infections in patients with non-infected dermatoses

| | Mean Total Score ( X̅ ± SD) | | | | | |
|---|---|---|---|---|---|---|
| | Indication | | | | | |
| Duration | Effect on Scaling | | Effect on Erythema | | Effect on Itching | |
| in days | Example 4 | Halbate ® | Example 4 | Halbate ® | Example 4 | Halbate ® |
| Baseline | 2.51 ± 0.89 | 2.47 ± 0.83 | 2.49 ± 0.98 | 2.32 ± 0.86 | 2.29 ± 0.70 | 2.21 ± 0.81 |
| 6-7 | *1.66 ± 0.72 | *1.86 ± 0.78 | *1.38 ± 0.77 | @*1.82 ± 0.68 | *1.07 ± 0.65 | @*1.67 ± 0.72 |
| 13-14 | *0.49 ± 0.57 | @*0.98 ± 0.85 | *0.41 ± 0.54 | @*0.91 ± 0.70 | *0.50 ± 0.67 | @*1.03 ± 0.66 |
| | Indication | | | | | |
| | Effect on | | | | | |
| Duration | Induration/Edema | | Effect on Pain | | Effect on Exudation | |
| in days | Example 4 | Halbate ® | Example 4 | Halbate ® | Example 4 | Halbate ® |
| Baseline | 2.11 ± 0.54 | 1.97 ± 0.77 | 2.31 ± 0.88 | 2.22 ± 0.73 | 1.83 ± 0.71 | 1.78 ± 0.78 |
| 6-7 | *1.01 ± 0.66 | @*1.34 ± 0.52 | *1.13 ± 0.75 | @*1.70 ± 0.65 | *0.92 ± 0.56 | @*1.40 ± 0.55 |
| 13-14 | *0.36 ± 0.49 | @*1.06 ± 0.49 | *0.46 ± 0.52 | @*0.95 ± 0.59 | *0.49 ± 0.53 | @*0.79 ± 0.48 |

By Wilcoxon Sign Rank Test *P < 0.05 Vs Basal, Significant
By Mann Whitney U Test @Between Groups P < 0.05 Significant

Table 1A indicates that after the end of treatment, mean score for scaling of 80.5% among patients treated with combination of Halobetasol and Fusidic Acid Cream, Example 4, which was significantly more as compared to those treated with 60.3% in Halobetasol Cream, Halbate. After the end of treatment, mean score of erythema, had a fall of 83.5% among patients treated with combination of Halobetasol and Fusidic Acid Cream, Example 4, which was significantly more as compared to 60.8% in patients treated with Halobetasol Cream, Halbate®. After the end of treatment, mean score of itching, had a fall of 78.2% among patients treated with combination of Halobetasol and Fusidic Acid Cream, Example 4, which was significantly more as compared to 53.4% in patients treated with Halobetasol Cream, Halbate®. After the end of treatment, mean score of induration/edema had a fall of 82.9% among patients treated with combination of Halobetasol and Fusidic Acid Cream, Example 4 which was significantly more as compared to 46.2% in patients treated with Halobetasol Cream, Halbate®. After the end of treatment, mean score for pain had a fall of 80.1% among patients treated with combination of Halobetasol and Fusidic Acid Cream, Example 4, which was significantly more as compared to 57.2% in patients treated with Halobetasol Cream. After the end of treatment, mean score for exudation had a fall of 73.2% among patients treated with combination of Halobetasol and Fusidic Acid Cream, Example 4, which was significantly more as compared to 55.6% in patients treated with Halobetasol Cream, Halbate®. After the end of treatment, mean score for total signs and symptoms had a fall of 80.9% among patients treated with combination of Halobetasol and Fusidic Acid Cream, Example 4, which was significantly more as compared to 55.9% in those treated with Halobetasol Cream, Halbate®

TABLE 2A
Table 2A: Results of changes in severity of lesions with combination of
Halobetasol and Fusidic Acid Cream (Example 4 of the present invention) vs
Halobetasol propionate 0.05% cream, Halbate ® in prevention of
secondary bacterial infections in patients with non-infected dermatoses.

| | Changes in Severity Of Lesions Between Two Groups | | | | | |
|---|---|---|---|---|---|---|
| | Example 4 | | | Halbate ® | | |
| | | Day | Day | | Day | Day |
| | Baseline | 6-7 | 13-14 | Baseline | 6-7 | 13-14 |
| | (N = 28) | (N = 28) | (N = 28) | (N = 27) | (N = 27) | (N = 27) |
| Severity | No. % | No. % | No. % | No. % | No. % | No. % |
| Mild | 04 (14.2) | 08 (28.6) | *22 (78.7) | 05 (18.6) | 06 (22.2) | 10 (37.0) |
| Moderate | 12 (42.9) | 13 (46.4) | 04 (14.2) | 11 (40.7) | 12 (44.5) | 11 (40.8) |
| Severe | 12 (42.9) | 07 (25.0) | 02 (07.1) | 11 (40.7) | 09 (33.3) | 06 (22.2) |

At the end of treatment 78.7% of total cases had mild severity of lesion in patients treated with combination of Halobetasol and Fusidic Acid Cream, Example 4, which was significantly more as compared to 37.0% among those treated with Halobetasol Cream, Halbate®

TABLE 3A
Table 3A: Results of changes in discharge of lesions with combination of
Halobetasol and Fusidic Acid Cream (Example 4 of the present invention) vs
Halobetasol propionate 0.05% cream, Halbate ® in prevention of
secondary bacterial infections in patients with non-infected dermatoses.

| | Changes in Discharge For Lesions Between Two Groups | | | | | |
|---|---|---|---|---|---|---|
| | Example 4 | | | Halbate ® | | |
| | | Day | Day | | Day | Day |
| | Baseline | 6-7 | 13-14 | Baseline | 6-7 | 13-14 |
| Type of | (N = 28) | (N = 28) | (N = 28) | (N = 27) | (N = 27) | (N = 27) |
| Discharge | No. % | No. % | No. % | No. % | No. % | No. % |
| Absent | 23 (82.2) | 24 (85.7) | *22 (78.5) | 23 (85.2) | 13 (48.2) | *06 (22.2) |
| Serous | 05 (17.8) | 04 (14.3) | 04 (14.3) | 04 (14.8) | 06 (22.2) | 10 (37.0) |
| Purulent | 00 (00) | 00 (00) | *01 (03.6) | 00 (00) | 6 (22.2) | *08 (29.7) |
| Mucopurulent | 00 (00) | 00 (00) | 01 (03.6) | 00 (00) | 02 (7.4) | 03 (11.1) |

By Chi - Square test *P < 0.05 Significant

Results showed that there was a significant decrease (p<0.05) in the mean symptom/sign scores as well as the total scores 1st week onwards and was significant sustained till two weeks (p<0.05) in patients treated with combination of Halobetasol propionate 0.05% w/w and Fusidic acid 2% w/w cream, Example 4 compared to patients treated with Halobetasol propionate 0.05% cream, Halbate®. In both the groups at baseline did not show purulent to mucopurulent type of discharge. At the end of treatment 7.2% of total cases had purulent to mucopurulent type of discharge in Patients treated with combination of Halobetasol propionate 0.05% w/w and Fusidic acid 2% w/w cream which was significantly low as compared to 40.8% among patients treated with Halobetasol propionate 0.05% w/w cream, Halbate® (p<0.05). 89.2-92.5% of the total cases had no significant bacteriological growth in both the groups at baseline and difference was not significant between the groups. The common bacteriological isolates were Staphylococcus aureus in both the groups. After the treatment only 7.2% of the cases had a significant growth among patients treated with combination of Halobetasol propionate 0.05% w/w and Fusidic acid 2% w/w cream, Example 4 which was significantly very low as compared to 55.6% in patients treated with Halbate® (p<0.05). According to Physician's global assessment, 78.6% of total cases showed complete resolution among total signs & symptoms in patients treated with combination of Halobetasol propionate 0.05% w/w and Fusidic acid 2% w/w cream, Example 4 group which was significantly more as compared to 26.0% among those treated with Halbate® (p<0.05). According to patients global evaluation, 78.6% of total cases had greatly improvement in combination of Halobetasol propionate 0.05% w/w and Fusidic acid 2% w/w cream, Example 4 group which was significantly more as compared to 25.9% among Halbate® group (p<0.05).

The formulations of the present invention were also assessed for efficacy, safety and tolerability of combination of Halobetasol 0.05% w/w and Fusidic acid 2% w/w cream, Example 4 in treatment of patients with infected dermatoses. Prospective, randomized, monocentre, open label study was conducted with 25 Male & female (post-menopausal, surgically sterilized or practicing a reliable method of birth control) patients with age ranging from 12 to 65 years with a clinical diagnosis of infected dermatoses with secondary bacterial infections confirmed by laboratory evaluation (gram stain & culture). Patients fulfilling the selection criteria were assigned to treatment with combination of Halobetasol propionate 0.05% w/w and Fusidic acid 2% w/w cream, Example 4, topically daily every 12 hours for 2 weeks after obtaining their informed consent. The duration of the study was 3 weeks including a 2-week week active treatment period preceded by a 1-week washout period. The efficacy variables included changes in mean scores of scaling, erythema, pruritus/itching, induration/edema, pain, exudation/crusting, total sign/symptom score, severity of lesion, discharge from lesions and overall global assessment of efficacy by physician/patients and monitoring of treatment-emergent adverse events. The study data was pooled and results were analyzed using a non-parametric test. All tests were two tailed & p<0.05 were considered to be significant.

TABLE 4A
Table 4A: Results of clinical symptoms with combination of Halobetasol
propionate 0.05% w/w and Fusidic acid 2% w/w cream, Example 4 in treatment
of patients with infected dermatoses

| | Mean Total Score ( X̅ ± SD) | | | | | |
|---|---|---|---|---|---|---|
| | Indication | | | | | |
| Duration | Effect on Scaling | | Effect on Erythema | | Effect on Itching | |
| in days | Example 4 | % change | Example 4 | % change | Example 4 | % change |
| Baseline | 2.39 ± 0.69 | — | 2.44 ± 0.79 | — | 2.30 ± 0.90 | — |
| 6-7 | *1.69 ± 0.53 | 29.3 | *1.19 ± 0.70 | 51.2 | *1.22 ± 0.64 | 46.9 |
| 13-14 | *0.33 ± 0.48 | 86.2 | *0.28 ± 0.50 | 88.5 | *0.49 ± 0.53 | 78.7 |
| | Indication | | | | | |
| | Effect on | | | | | |
| Duration | Induration/Edema | | Effect on Pain | | Effect on Exudation | |
| in days | Example 4 | % change | Example 4 | % change | Example 4 | % change |
| Baseline | 2.13 ± 0.75 | — | 2.33 ± 0.83 | — | 1.61 ± 0.74 | — |
| 6-7 | *1.12 ± 0.66 | 47.4 | *1.21 ± 0.67 | 48.1 | *0.85 ± 0.49 | 47.2 |
| 13-14 | *0.51 ± 0.59 | 76.1 | *0.42 ± 0.60 | 81.9 | *0.36 ± 0.48 | 77.6 |

By Wilcoxon Sign Rank Test *P < 0.05 Vs Basal, Significant

In this study, the mean total score of symptoms were 13.20 at baseline. After the treatment at the end of 6-7 days mean score had a significant reduction i.e. 44.8%. At the end of 13-14 days mean score had a fall of 81.9% from baseline.

TABLE 5A
Results of changes in severity of lesions for combination of
Halobetasol propionate 0.05% w/w and Fusidic acid 2% w/w cream,
Example 4 in treatment of patients with infected dermatoses
Table 5A: Results of changes in severity of lesions

| | | |
|---|---|---|
| Baseline | Day 6-7 | Day 13-14 |
| (N = 28) | (N = 28) | (N = 28) |

| Severity | No. | % | No. | % | No. | % |
|---|---|---|---|---|---|---|
| Mild | 05 | 17.9 | *09 | 32.1 | *23 | 82.1 |
| Moderate | 10 | 35.7 | *13 | 46.5 | 03 | 10.8 |
| Severe | 13 | 46.4 | 06 | 21.4 | 02 | 07.1 |

By Chi - Square Test *P < 0.05 Vs Baseline, Significant

Table 5A shows that 53.6% of the total study cases had a mild to moderate severity of lesions at baseline. After treatment at the end of 6-7 days, 78.6% of the total cases showed mild to moderate severity of lesions which was considerable change and difference was significant. At the end of treatment 82.1% of the cases had a mild severity of lesions which was significant change from baseline.

TABLE 6A
Results of changes in discharge of lesions for combination
of Halobetasol propionate 0.05% w/w and Fusidic acid 2% w/w cream,
Example 4 in treatment of patients with infected dermatoses.
Table 6A: Results of changes in discharge of lesions

| | Day 3 | | Day 6-7 | | Day 13-14 | |
|---|---|---|---|---|---|---|
| | (N = 28) | | (N = 28) | | (N = 28) | |
| Type of Discharge | No. | % | No. | % | No. | % |
| Absent | — | — | 07 | 25.0 | *19 | 67.9 |
| Serous | 06 | 21.4 | 11 | 39.3 | 05 | 17.9 |
| Purulent | 15 | 53.6 | *07 | 25.0 | 02 | 07.1 |
| Mucopurulent | 07 | 25.0 | *03 | 10.7 | 02 | 07.1 |

By Chi - Square Test *P < 0.05 Vs Baseline, Significant

Table 6A indicates, 78.6% of the total study cases had a purulent to mucopurulent type of discharge for lesions at baseline. After treatment at the end of 6-7 days, only 35.7% of the total cases showed same type of discharge for lesions which was statistically significant from baseline. At the end of treatment 67.9% of the cases did not had a discharge and 17.9% serous discharge after treatment with Example 4.

Results showed that there was a significant decrease (p<0.05) in the mean symptom/sign scores as well as the total scores 1st week onwards and was significant sustained till two weeks (p<0.05) in case of treatment with combination of Halobetasol 0.05% w/w and Fusidic acid 2% w/w cream, Example 4 in treatment of patients with infected dermatoses. 82.1% of the total cases had significant bacteriological growth at baseline. Out of which 46.4% had a Staphylococcus aureus, 1.4% Streptococcus Spp and 7.1% Proteus Spp. After the treatment only 10.7% of the cases had a growth which was significantly low from baseline. There was a significant decrease (89.3%) in bacteriological growth compared to baseline (P<0.05). Combination of Halobetasol propionate 0.05% w/w and Fusidic acid 2% w/w cream of the present invention proved to be effective in 100% cases with 92.9% cases showing marked improvement to complete resolution of symptoms, the remaining 7.1% cases also showed moderate improvement. A total of 6.7% of total cases had an adverse event. Out of this each one of them had a burning, irritation and dry skin. The severity of events was mild to moderate in both the cases which were disappeared during the treatment. Combination of Halobetasol propionate 0.05% w/w and Fusidic acid 2% w/w cream of the present invention proved to be effective in 100% cases with 92.9% cases showing marked improvement to complete resolution of symptoms, the remaining 7.1% cases also showed moderate improvement.

This is superior to the results obtained by Javier P R et al 1986 (Ref: Javier P R, Ortiz M, Torralba L, Montinola F L, Ke M L, Canete R. Fusidic acid/betamethasone in infected dermatoses—a double-blind comparison with neomycin/betamethasone. Br J Clin Pract. 1986; 40:235-8), with the fixed dose combination of betamethasone 0.1% w/w and fusidic acid 2% w/w cream, where efficacy was seen in 85% cases. In a study of Halobetasol propionate 0.05% w/w by Kantor et al 1991 (Ref: Kantor I, Cook P R, Cullen S I, et al. Double-blind bilateral paired comparison of 0.05% halobetasol propionate cream and its vehicle in patients with chronic atopic dermatitis and other eczematous dermatoses. J Am Acad Dermatol 1991 December; 25(6 Pt 2):1184-6), in patients of atopic dermatitis and other eczematous dermatoses, showed excellent to good clinical improvement in 87% cases. Combination of Halobetasol propionate 0.05% w/w and Fusidic acid 2% w/w cream of the present invention also proved to be effective in eradication of the bacterial organisms in 89.3% of cases. This was found to be superior to the results obtained by Hjorth N, et al 1985 (Ref: Hjorth N, Schmidt H, Thomsen K. Fusidic acid plus betamethasone in infected or potentially infected eczema. Pharmatherapeutica. 1985; 4:126-31), where bacteriological cures were 67% and by Javier P R et al 1986, where bacteriological cures were 78%. Therapy with the combination of Halobetasol propionate 0.05% w/w and Fusidic acid 2% w/w cream of the present invention provided a fast, better efficacy and safe tolerability.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

1-22. (canceled)

23. A topical pharmaceutical composition comprising:
a) fusidic acid in the range of 1% w/w to 5% w/w; and
b) a corticosteroid selected from mometasone and halobetasol or their esters in the range of 0.01% w/w to 2% w/w, based on total weight of the composition.

24. The topical pharmaceutical composition according to claim 23, wherein the composition comprises fusidic acid in the range of 1% w/w to 5% w/w and mometasone furoate in the range of 0.05% w/w to 2% w/w.

25. The topical pharmaceutical composition according to claim 23, wherein the composition comprises 2% w/w fusidic acid, 0.1% w/w mometasone furoate and a pharmaceutically acceptable carrier.

26. The topical pharmaceutical composition according to claim 23, wherein the weight ratio of mometasone furoate to fusidic acid ranges from 1:2.5 to 1:20.

27. The topical pharmaceutical composition according to claim 23, wherein the weight ratio of mometasone furoate to fusidic acid ranges from 1:10 to 1:20.

28. A method for the treatment or prevention of infected eczemas including secondarily infected dermatitis, allergic contact dermatitis, atopic dermatitis, and psoriasis in a mammal, said method comprising topically applying to the mammal a pharmaceutical composition comprising fusidic acid in the range of 1% w/w to 5% w/w and mometasone furoate in the range of 0.05% w/w to 2% w/w.

29. The method according to claim 28, wherein the composition comprises 2% w/w fusidic acid and 0.1% w/w mometasone furoate.

30. The topical pharmaceutical composition according to claim 23, wherein the composition comprises fusidic acid in the range of 1% w/w to 5% w/w and halobetasol propionate in the range of 0.01% w/w to 2% w/w.

31. The topical pharmaceutical composition according to claim 23, wherein the composition comprises 2% w/w fusidic acid, 0.05% w/w halobetasol propionate and a pharmaceutically acceptable carrier.

32. The topical pharmaceutical composition according to claim 23, wherein the weight ratio of halobetasol propionate to fusidic acid ranges from 1:2.5 to 1:100.

33. The topical pharmaceutical composition according to claim 23, wherein the weight ratio of halobetasol propionate to fusidic acid ranges from 1:30 to 1:50.

34. A method for the treatment of steroid responsive infected dermatoses including secondarily infected contact dermatitis, allergic contact dermatitis, atopic dermatitis, and psoriasis in a mammal, said method comprising topically applying to the mammal a pharmaceutical composition comprising fusidic acid in the range of 1% w/w to 5% w/w and halobetasol propionate in the range of 0.01% w/w to 2% w/w, based on total weight of the composition.

35. The method according to claim 34, wherein the composition comprises 2% w/w fusidic acid and 0.05% w/w halobetasol propionate.

36. A method for the prevention of secondary bacterial infections in patients with non-infected dermatoses in a mammal, said method comprising topically applying to the mammal a pharmaceutical composition fusidic acid in the range of 1% w/w to 5% w/w and halobetasol propionate in the range of 0.01% w/w to 2% w/w, based on total weight of the composition.

37. The method according to claim 36, wherein the composition comprises 2% w/w fusidic acid and 0.05% w/w halobetasol propionate.
